(19) United States
(12) Patent Application Publication
(10) Pub. No.: US 2016/0206783 A1
(43) Pub. Date: Jul. 21, 2016

(54) CROSS-LINKED PLATELET MATERIAL

(71) Applicant: MAYO FOUNDATION FOR MEDICAL EDUCATION AND RESEARCH, Rochester, MN (US)

(72) Inventors: Allan B. DIETZ, Rochester, MN (US); Gaylord J. KNUTSON, Rochester, MN (US)

(21) Appl. No.: 14/914,574

(22) PCT Filed: Aug. 26, 2014
(86) PCT No.: PCT/US2014/052683

§ 371 Date: Feb. 25, 2016

Related U.S. Application Data
(60) Provisional application No. 61/870,523, filed on Aug. 27, 2013.

(51) Int. Cl. A61L 27/36; A61L 27/22; A61L 26/00; A61L 27/54

(52) U.S. Cl. CPC A61L 27/3616; A61L 27/3687; A61L 27/54; A61L 27/227; A61L 27/3691; A61L 26/0057; A61L 26/0066; A61L 26/0047; A61L 2300/414; A61L 2430/22

(57) ABSTRACT

This document provides methods and materials involved in making and using cross-linked platelet material (e.g., cross-linked lysate material from human platelets such as human platelets obtained from platelet blood collection preparations or platelet apheresis preparations). For example, methods and materials for cross-linking platelet material (e.g., lysate material obtained from human platelets) to form a matrix (e.g. a cell-free tissue scaffold) for wound healing or regenerative medicine or to form conjugates or modified molecules are provided.

### CROSS-REFERENCE TO RELATED APPLICATIONS

This application claims the benefit of U.S. Provisional Application Ser. No. 61/870,523, filed Aug. 27, 2013. The disclosure of the prior application is considered part of (and is incorporated by reference in) the disclosure of this application.

### BACKGROUND

1. Technical Field

This document relates to methods and materials involved in making and using cross-linked platelet material (e.g., cross-linked lysate material from human platelets such as human platelets obtained from platelet blood collection preparations or platelet apheresis preparations). For example, this document relates to methods and materials for cross-linking platelet material (e.g., lysate material obtained from human platelets) to form a matrix (e.g. a cell-free tissue scaffold) for wound healing or regenerative medicine.

2. Background Information

Matrices such as biocompatible and biodegradable matrices can be used to facilitate tissue growth, cell proliferation, and cell infiltration to repair or regenerate tissue. For example, naturally-occurring and synthetic biodegradable materials can be designed as scaffolds for tissue repair.

### SUMMARY

This document relates to methods and materials involved in making and using cross-linked platelet material (e.g., cross-linked lysate material from human platelets such as human platelets obtained from platelet blood collection preparations or platelet apheresis preparations). For example, this document provides methods and materials for cross-linking platelet material (e.g., lysate material obtained from human platelets) to form a matrix (e.g. a cell-free tissue scaffold) for wound healing or regenerative medicine or to form conjugates or modified molecules.

As described herein, platelet material (e.g., platelet lysate material) can be cross-linked to a molecule such as genipin, gluteraldehyde, or alginate dialdehyde (AD) to create gel-like material or conjugate or can be cross-linked to a matrix via a molecule such as genipin, AD, or gluteraldehyde to create a matrix containing platelet material (e.g., platelet lysate material). In some cases, the cross-linked platelet material (e.g., platelet lysate material), whether cross-linked to a molecule such as genipin, gluteraldehyde, or AD to create a gel-like material or cross-linked to a matrix via a molecule such as genipin, gluteraldehyde, or AD, can maintain the ability to promote cell growth, cell proliferation, and/or cell infiltration (e.g., infiltration into the gel-like material or matrix).

In some cases, intact platelets can be cross-linked via a molecule such as genipin, AD, or gluteraldehyde to create a cross-linked platelet preparation. Such a preparation can be used as is with the intact platelets or can be used following lysis of the platelets. For example, a cross-linked platelet preparation can be obtained using intact platelets, then lysed to create a preparation that includes platelet material cross-linked to other platelet material.

In general, one aspect of this document features a composition comprising, or consisting essentially of, platelet material cross-linked to genipin. The platelet material can be a lysed platelet preparation. The lysed platelet preparation can be a preparation that was filtered through a 0.45 μm filter. The lysed platelet preparation can be a preparation that was filtered through a 0.2 μm filter. The lysed platelet preparation can be a preparation that was filtered through a 0.45 μm filter and a 0.2 μm filter. The platelet material can comprise supernatant from centrifugation of lysed platelets. The platelet material can be platelets lysed via a freeze/thaw cycle. The lysed platelets can be platelets lysed via at least two freeze/thaw cycles. The centrifugation can comprise a force between 2000×g and 4000×g for between 15 and 45 minutes. The centrifugation can comprise a force of about 3000×g for about 30 minutes. The platelet material can comprise greater than 200 pg of VEGF polypeptide per mL. The platelet material can comprise from about 20 mg to about 80 mg of total protein per mL (e.g., from about 30 mg to about 80 mg of total protein per mL, from about 40 mg to about 80 mg of total protein per mL, from about 50 mg to about 80 mg of total protein per mL, from about 20 mg to about 70 mg of total protein per mL, from about 20 mg to about 60 mg of total protein per mL, from about 30 mg to about 70 mg of total protein per mL, or from about 50 mg to about 60 mg of total protein per mL). The composition can be prepared by combining the platelet material with the genipin. The composition can be prepared by combining a solution of between 5 percent and 100 percent of the platelet material with the genipin. The composition can be prepared by combining a solution of between 60 percent and 95 percent of the platelet material with the genipin. The composition can be prepared by combining a solution of between 80 percent and 100 percent of the platelet material with the genipin. The composition can be prepared by combining the platelet material with from about 1.5 mg to about 20 mg of the genipin per mL. The composition can be prepared by combining the platelet material with from about 2.5 mg to about 10 mg of the genipin per mL. The composition can be prepared by combining the platelet material with from about 2.0 mg to about 5.0 mg of the genipin per mL. The composition can be configured in the shape of a film or sheet. The thickness of the composition can be between 50 μm and 10 mm (e.g., between 50 μm and 10 mm, between 50 μm and 5 mm, between 50 μm and 1 mm, between 50 μm and 0.1 mm, between 75 μm and 10 mm, between 100 μm and 10 mm, between 250 μm and 10 mm, or between 500 μm and 5 mm).

In another aspect, this document features a composition comprising, or consisting essentially of, platelet material attached to a matrix, wherein genipin cross-links the platelet material to a surface of the matrix. The platelet material can be a lysed platelet preparation. The lysed platelet preparation can be a preparation that was filtered through a 0.45 μm filter. The lysed platelet preparation can be a preparation that was filtered through a 0.2 μm filter. The lysed platelet preparation can be a preparation that was filtered through a 0.45 μm filter and a 0.2 μm filter. The platelet material can comprise supernatant from centrifugation of lysed platelets. The platelet material can be platelets lysed via a freeze/thaw cycle. The lysed platelets can be platelets lysed via at least two freeze/thaw cycles. The centrifugation can comprise a force between 2000×g and 4000×g for between 15 and 45 minutes. The centrifugation can comprise a force of about 3000×g for about 30 minutes. The platelet material can comprise greater than 200 pg of VEGF polypeptide per mL. The platelet material can comprise from about 20 mg to about 80 mg of total protein per mL (e.g., from about 30 mg to about 80 mg of total protein per mL, from about 40 mg to about 80 mg of total protein per mL, from about 50 mg to about 80 mg of total protein per mL, from about 20 mg to about 70 mg of total protein per mL, from about 20 mg to about 60 mg of total protein per mL, from about 30 mg to about 70 mg of total protein per mL, or from about 50 mg to about 60 mg of total protein per mL). The composition can be prepared by combining the platelet material with the matrix, wherein the matrix is coated with the genipin. The composition can be prepared by combining a solution of between 5 percent and 100 percent of the platelet material with the matrix, wherein the matrix is coated with the genipin. The composition can be prepared by combining a solution of between 60 percent and 95 percent of the platelet material with the matrix, wherein the matrix is coated with the genipin. The composition can be prepared by combining a solution of between 80 percent and 100 percent of the platelet material with the matrix, wherein the matrix is coated with the genipin. The composition can be prepared by combining the platelet material with from about 1.5 mg to about 20 mg of the genipin per mL to form a mixture, and contacting the mixture to the matrix. The composition can be prepared by combining the platelet material with from about 2.5 mg to about 10 mg of the genipin per mL to form a mixture, and contacting the mixture to the matrix. The composition can be prepared by combining the platelet material with from about 2.0 mg to about 5.0 mg of the genipin per mL to form a mixture, and contacting the mixture to the matrix. The composition can be configured in the shape of an esophageal segment. The composition can be a bandage for wound healing, an implantable esophageal segment, or a mucosal replacement device.

In another aspect, this document features a composition comprising, or consisting essentially of, a method for making a composition comprising platelet material and genipin, wherein the method comprises contacting the platelet material with genipin, wherein the platelet material cross-links to the genipin.

In another aspect, this document features a composition comprising, or consisting essentially of, a method for making a composition comprising a matrix, platelet material, and genipin, wherein the method comprises attaching the platelet material to the matrix via genipin.

In another aspect, this document features a composition comprising, or consisting essentially of, a method for repairing an esophagus within a mammal, wherein the method comprising implanting an tubular tissue scaffold into the mammal in a position that bridges a gap in the esophagus, wherein the tubular tissue scaffold comprises platelet material. The tubular tissue scaffold can comprise genipin. The platelet material can be cross-linked to the tubular tissue scaffold via genipin. The tubular tissue scaffold can comprise nanofibers.

In another aspect, this document features a composition comprising, or consisting essentially of, a method for healing a wound, wherein the method comprising contacting the wound with a matrix comprising platelet material attached to the matrix via genipin. The matrix can be a bandage.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagram of a scaffold containing cross-linked platelet material configured for use as a mucosal replacement device. The muscularis mucosa and epithelium are removed and replaced with a scaffold containing cross-linked platelet material according some embodiments.

FIG. 2A is a photograph of mesenchymal stormal cells (MSCs) cultured with media plus 0% platelet lysate material. FIG. 2B is a photograph of MSCs cultured with media plus 5% platelet lysate material.

FIG. 3A is a photograph of MSCs cultured with cross-linked platelet lysate material using 60% of platelet lysate material in combination with media containing 0% platelet lysate material. FIG. 3B is a photograph of MSCs cultured with cross-linked platelet lysate material using 80% of platelet lysate material in combination with media containing 0% platelet lysate material. FIG. 3C is a photograph of MSCs cultured with cross-linked platelet lysate material using 100% of platelet lysate material in combination with media containing 0% platelet lysate material.

### DETAILED DESCRIPTION

This document provides methods and materials involved in making and using cross-linked platelet material (e.g., cross-linked lysate material from human platelets such as human platelets obtained from platelet blood collection preparations or platelet apheresis preparations). For example, this document provides methods and materials for cross-linking platelet material (e.g., lysate material obtained from human platelets) to form a matrix (e.g. a cell-free tissue scaffold) for wound healing or regenerative medicine or to form conjugates or modified molecules.

Any appropriate source of platelets can be used to make a composition containing cross-linked platelet material (e.g., cross-linked platelet lysate material). For example, apheresis platelets and platelets derived from normal blood donation can be used as a source of platelets for making cross-linked platelet material (e.g., cross-linked platelet lysate material).

In one embodiment, platelet material can be obtained as follows. Once platelets are obtained, the platelets can be used intact or can be treated with any appropriate method to release the content of the platelets including, without limitation, a single freeze/thaw cycle, repeated (e.g., 2, 3, 4, 5, or more) freeze/thaw cycles, detergent lysis, activation with thrombin, collagen, thromboxane A2, ADP or other factors, and manipulation of ionic strength. In some cases, two freeze/thaw cycles can be used to obtain platelet lysate material. Once lysed, the lysed platelet preparation can be centrifuged to obtain a supernatant. In general, the force of centrifugation can be between 1000×g and 10000×g (e.g., between 1000×g and 7500×g, between 1000×g and 5000×g, between 1000×g and 2500×g, between 2000×g and 5000×g, between 3000×g and 5000×g, between 4000×g and 5000×g, or between 2000×g and 5000×g), and the duration can be between 5 minutes and 3 hours (e.g., between 5 minutes and 120 minutes, between 10 minutes and 120 minutes, between 5 minutes and 60 minutes, between 10 minutes and 60 minutes, or between 15 minutes and 45 minutes). For example, a lysed platelet preparation can be centrifuged at about 3,000×g for about 30 minutes. Once the supernatant is collected, it can be filtered. For example, the supernatant can be filtered through a 0.45 μm filter, a 0.2 μm filter, or a 0.45 μm filter followed by a 0.2 μm filter. The resulting filtrate can be used as platelet lysate material without further processing or can be combined with heparin to form heparin-treated platelet lysate material.

In some cases, platelet lysate material provided herein can be prepared without washing the platelets prior to lysing them. In such cases, the platelet lysate material can include plasma and plasma components. For example, platelet lysate material provided herein can include albumin and/or thrombin at about physiologic concentrations. In some cases, platelet lysate material provided herein can include platelet contents prepared from platelets lysed in the presence of plasma or a plasma-like composition.

In some cases, platelet material (e.g., platelet lysate material) provided herein can lack recombinant polypeptides, can lack recombinant nucleic acid, or can lack both recombinant polypeptides and recombinant nucleic acid.

In some cases, platelet material (e.g., platelet lysate material) provided herein can contain between about 35 mg to about 75 mg of protein per mL (e.g., between about 35 mg to about 70 mg of protein per mL, between about 35 mg to about 65 mg of protein per mL, between about 35 mg to about 60 mg of protein per mL, between about 40 mg to about 75 mg of protein per mL, between about 45 mg to about 75 mg of protein per mL, or between about 50 mg to about 60 mg of protein per mL). In some cases, platelet material (e.g., platelet lysate material) provided herein can contain about 55 mg/mL of protein. Additional characteristics of platelet material (e.g., platelet lysate material) that can be used as described herein are described elsewhere (Crespo-Diaz et al., Cell Transplantation, 20(6):797-811 (2011)).

In another embodiment, platelet lysate material can be obtained as follows. Platelets can be maintained between 2° C. and 42° C. (e.g., between 2° C. and 40° C., between 2° C. and 38° C., between 2° C. and 36° C., between 2° C. and 30° C., between 5° C. and 36° C., between 10° C. and 36° C., between 15° C. and 36° C., between 20° C. and 30° C.) for a period of time (e.g., two, three, four, five, or more days) in the presence of plasma without performing an active step designed to lyse the platelets. For example, a platelet preparation (e.g., outdated platelet preparation) obtained from an apheresis technique can be used without removing the plasma. Once obtained, the platelet lysate material can be treated to remove platelets, platelet debris, or platelet ghosts to obtain the resulting medium that includes platelet lysate material and plasma components. For example, this resulting medium can be obtained by centrifugation and/or filtration. Once obtained, the resulting medium containing platelet lysate material can be stored or used as platelet lysate material as described herein.

In some cases, platelet material (e.g., platelet lysate material) provided herein can be cross-linked or attached to a molecule. Examples of such molecules include, without limitation, genipin, AD, clotting factors, calcium, thrombin, or gluteraldehyde. Compositions containing platelet material (e.g., platelet lysate material) cross-linked to a molecule such as genipin can be used to coat a matrix such as polyglycolic acid, polylactic acid, polydioxanone, and caprolactone. In some cases, cross-linking platelet material (e.g., platelet lysate material) provided herein to a molecule such as genipin can result in a film or sheet. Such films or sheets can be used to provide a source of platelet material (e.g., platelet lysate material). For example, a film or sheet of platelet material (e.g., platelet lysate material) cross-linked to genipin can be applied to a wound to assist in the healing process. In some cases, a film or sheet of platelet material (e.g., platelet lysate material) cross-linked to genipin can dried to form a dried film or sheet of platelet material (e.g., platelet lysate material) cross-linked to genipin. Such dried films or sheets can be stored until ready for use.

In some cases, films can be combined with different properties (some bound with platelet lysate material and others without or with other signaling properties) to form a matrix composition with multiple signals or complex properties.

In some cases, platelet material (e.g., platelet lysate material) provided herein can be cross-linked or attached to a matrix via genipin, AD, clotting factors, calcium, thrombin, or gluteraldehyde. Examples of such matrices include, without limitation, wound care devices such as stitches, bandages, and wound patches, implantable medical devices such as esophageal segments, stents, scaffolds, joint replacements, and valves, and tissue filler devices such as platinum coils, cartilage binding elements, tendons, and bone extracts of purified epithelium from human or xenogeneic sources. In some cases, a matrix can be coated with genipin, and platelet material (e.g., platelet lysate material) can be cross-linked to the genipin. In some cases, a matrix provided herein containing platelet material (e.g., platelet lysate material) can be free of cells.

In some cases, a molecule such as genipin can be used with platelet material (e.g., platelet lysate material) in a controlled manner such as in 3-dimensional matrix printing. Such matrix printing can be used with or without cells to develop specific layers of growth inducing cross-linked platelet material (e.g., platelet lysate material). In some cases, cells (e.g., mesenchymal stomal cells, endothelial cells, stem cells, epithelial cells, or primary organ derived cells) can be pre-seeded during the 3D printing. In some cases, growth factor elements can be simultaneously embedded in a manner to direct cell division or cell lineages (e.g., endothelial vs. mesenchymal cell growth).

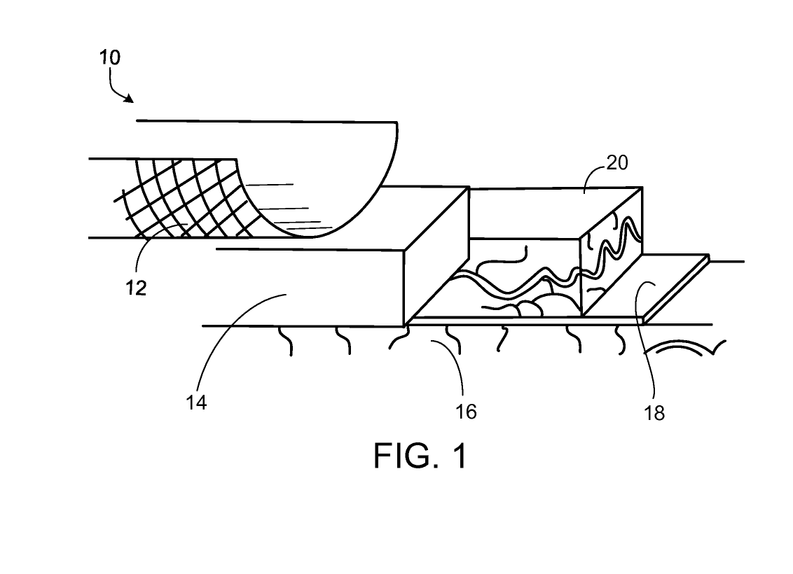

With reference to FIG. 1, a mucosal replacement device 10 can be configured to include a stent 12 and a scaffold 14 containing cross-linked platelet material (e.g., platelet lysate material). In some cases, scaffold 14 can be composed of platelet material (e.g., platelet lysate material) cross-linked with genipin. Any appropriate stent configuration can be used. For example, a cylindrical stent can be used. In such cases, scaffold 14 can be cylindrical as well. As shown in FIG. 1, mucosal replacement device 10 can be configured such that scaffold 14 contacts submucosa 16 after muscularis mucosa 18 and epidermis 20 are removed. Any appropriate method can be used to remove muscularis mucosa 18 and epidermis 20 prior to implanting mucosal replacement device 10. For example, endoscopic mucosal resection, endoscopic submucosal dissection, esophageal mucosectomy, and/or ablation techniques can be used to remove sections of muscularis mucosa 18 and epidermis 20 prior to implanting mucosal replacement device 10.

The invention will be further described in the following examples, which do not limit the scope of the invention described in the claims.

### EXAMPLES

### Example 1

### Source of Platelets

All donors donating apheresis platelets fulfilled eligibility criteria as defined by AABB Standards for Blood Banks and Transfusion Service and the Food and Drug Administration. Donors were screened using the Uniform Donor History Questionnaire (UDQ) and accompanying educational materials. This questionnaire is a screening document created by a coalition of regulatory, accrediting, and blood collecting institutions consisting of the Food and Drug Administration, Centers for Disease Control and Prevention, Armed Services Blood Program, National Heart Lung and Blood Institute, American Blood Resources Association, AABB, American Red Cross, and America's Blood Centers. Information concerning the UDQ can be found on the World Wide Web at “fda.gov/cber/dhq/dhq.htm.”

All apheresis platelet donations were tested with the following infectious disease tests: (1) Serologic test for syphilis; (2) HCV EIA-hepatitis C virus antibody test, (3) HCV NAT-hepatitis C virus nucleic acid test, (4) HbsAg-hepatitis B surface antigen test, (5) Anti-HBc-hepatitis B Core antibody, (6) HIV-1/2 EIA-Human Immunodeficiency Virus 1/2 antibody test with ability to detect HIV 1 subgroup O; (7) HIV NAT-Human Immunodeficiency Virus nucleic acid test, (8) HTLV I/II EIA-Human T-Lymphotrophic Virus Types I/II, (9) WNV NAT-West Nile Virus nucleic acid test, and (10) Anti-T. cruzi, (serologic test for Chagas disease) using FDA licensed procedures.

In addition to the above tests, all apheresis platelet products were tested for bacterial contamination. Twenty-four hours after collection, the product was resuspended, and an 8 mL sample was collected. Four mL of this sample was inoculated into an anaerobic culture bottle, and 4 mL was inoculated into an aerobic bottle. These bottles were then placed into a BacT/ALERT® system (bioMérieux, Durham, N.C., USA) within three hours of inoculation and monitored for CO₂ generation for 24 hours. If after 24 hours CO₂ production was not detected, the platelet products were released and made available for transfusion. The culture bottles continued to be monitored for the remaining shelf-life of the platelet product (total of five days; three additional days after release).

The platelet products that were released for manufacture of platelet lysate material were collected from donors who fulfill donation criteria, were negative tests for the infectious diseases listed above, and exhibited no evidence of bacterial growth by their expiration date. Products from donors who failed to meet donor criteria, that exhibited positive infectious disease testing, or that produced cultures positive for bacteria were considered biohazardous waste. These products were quarantined and destroyed. They were not released for manufacture of platelet lysate material. FDA tests and guidelines for release can change. However, platelets used for these purposes met FDA tests and guidelines current at the time of production.

### Example 2

### Preparing Platelet Lysate from Apheresis Platelets

Apheresis platelets were obtained as described in Example 1. The apheresis platelets used were no more than four days past expiration. A single lot of platelet lysate consisted of at least ten individual apheresis platelet units, and one lot was used at a time to create a platelet lysate product. The processing for clinical grade reagents can be performed in a clean room suite. Platelet units were frozen at −70° C. or colder. After being frozen for at least 24 hours, the units were removed from the freezer and allowed to thaw. The units were thawed at room temperature or at refrigerated temperatures. Each thawed platelet bag was placed flat (to minimize breakage of tubing) in a freezer for a second freeze. After the apheresis platelet units were frozen for at least 24 hours for a second freeze, they were removed from the freezer and allowed to thaw. After the second thaw, the platelet product was centrifuged for 30 minutes at 3000×g for 30 minutes at room temperature using a Benchtop Centrifuge Sorvall Legend T. The resulting supernatants were transferred to 0.45-micron filter units (Pall Stericup, Catalog Number SCHV U05 RE; East Hills, N.Y. or Nalgene Filter System, Catalog Number 167-0045; Rochester, N.Y.). The filter unit was connected to a vacuum source and allowed to filter the product. If the product did not filter completely, the unfiltered product was transferred to another filter unit. The filtrates from all of the 0.45-micron filter units were pooled and filtered through a 0.2-micron filter unit (Pall Stericup, Catalog Number SCHV U05 RE; East Hills, N.Y. or Nalgene Filter System, Catalog Number 567-0020; Rochester, N.Y.). The filter unit was connected to a vacuum source and allowed to filter the product. If the product did not completely filter, the unfiltered product was transferred to a second filter unit, and the process was repeated as needed. The 0.2-micron filtrates were combined into receiver bottles or 2 L bags. The contents were mixed well. Heparin (1000 U/mL) was added to the filtered platelet lysates to obtain a final concentration of 2 U/mL.

The lysates were divided into aliquots. The lysates were stored frozen at ≦20° C. or colder.

One of the aliquots containing the platelet lysate was used to perform the following tests to determine whether or not to release the platelet lysate preparation for use:

Aerobic Culture.

One mL of platelet lysate was transferred to a Peds Bactec blood culture bottle (Becton, Dickinson and Company; Sparks, Md.) that was used to test sterility.

Anaerobic Culture.

Eight mL of platelet lysate was transferred to a Bactec Lytic/10 Anaerobic/F bottle (Becton, Dickinson and Company; Sparks, Md.). Briefly, both aerobic and anaerobic bottles are loaded in the BACTEC 9240 instrument (Becton, Dickinson and Company; Sparks, Md.) and monitored every four hours for 14 days. After 14 days, negative cultures are reported out as “No growth at 14 days”, positive cultures are subcultured and isolates identified.

Endotoxin Assay.

One mL of platelet lysate was transferred to a sterile endotoxin-free tube that was used to perform an endotoxin assay. Briefly, a 1:50 dilution of Platelet Lysate to Limulus Amebocyte Lysate (LAL) Reagent Water was run on the Endosafe Portable Test System (PTS; Charles River, Wilmington, Mass.). The Endosafe PTS utilizes LAL kinetic chromogenic methodology to measure color intensity directly related to the endotoxin concentration in a sample. Each disposable cartridge contains precise amounts of licensed LAL reagent, chromogenic substrate, and control standard endotoxin. The result obtained from each batch of Platelet Lysate must be <0.500 Endotoxin Units (EU)/mL.

Cell Kinetics.

A batch (≧150 mL) of Platelet Lysate-5% (PL5%) media containing Advanced-MEM (120 mL), GlutaMAX (1.2 mL), Heparin (˜0.24 mL), and 5% platelet lysate (6.4 mL) was prepared. A vial of previously frozen mesenchymal stem cells (reference cells) was thawed in a 37° C. water bath. Once thawed, the cells were placed in a sterile 50 mL tube with about 5 mL of the PL5% media. The tube was spun at 240×g for 5 minutes. The supernatant was removed from the tube, and one mL of the PL5% media was added to the cell pellet. A cell count was performed. The thawed cells were placed in one to two 175 cm² flasks with 50 mL of PL5% so that each flask contained 1.75×10⁵−4.38×10⁵ of the thawed cells. The flasks were incubated at 37° C. in a 5 percent CO₂ incubator. The cells were passaged using TrypLE™ (Invitrogen Corporation, Carlsbad, Calif.) after the flasks were confluent. The cells were combined, and a cell count performed. A population doubling calculation was performed.

Flow Cytometry.

The cells from the cell kinetic assay were assessed using the following flow cytometry panel:

| Tube # | FITC | PE |
|---|---|---|
| 1 | IgG1 | IGg1 |
| 2 | IgG2 | IgG2 |
| 3 | CD90 | CD73 |
| 4 | CD105 | HLA-DR |
| 5 | CD44 | HLA-ABC |
| 6 | CD45 | CD14 |

Platelet lysates that were sterile, endotoxin-free, grew MSC with the expression profile of CD105, CD90, CD73, HLA-ABC positive and negative for CD14, CD45, and HLA-DR were released for clinical use and assigned an expiration date of two years from production.

### Example 3

### Culturing Cells with Platelet Lysate Material Cross-Linked to Genipin

The platelet lysate material was prepared as described in Example 2. The growth medium was advanced MEM, 2 units/mL heparin, pen/strep, and either 0% or 5% platelet lysate material. When completed, complete platelet lysate contained of 55 mg/mL of protein (mean 95% confidence interval 48-62 mg/mL) and the components set forth in Table 1.

TABLE 1

| | | | | | | | Protein |
|---|---|---|---|---|---|---|---|
| | TGF B | IGF1 | EGF | PDGF | VEGF | FGF | mg/mL |
| Number of values | 9 | 9 | 9 | 9 | 9 | 9 | 9 |
| Minimum | 113.6 | 90.00 | 16.60 | 4.191 | 286.0 | 76.00 | 34.75 |
| 25% Percentile | 117.8 | 111.3 | 16.95 | 7.595 | 333.0 | 148.0 | 50.89 |
| Median | 129.4 | 131.4 | 17.50 | 9.125 | 498.0 | 184.0 | 58.91 |
| 75% Percentile | 134.8 | 149.8 | 20.90 | 11.58 | 609.5 | 220.0 | 61.35 |
| Maximum | 149.2 | 155.2 | 22.00 | 13.79 | 675.0 | 315.0 | 63.06 |
| Mean | 128.3 | 129.5 | 18.59 | 9.326 | 470.0 | 186.7 | 55.44 |
| Std. Deviation | 11.18 | 23.07 | 2.094 | 2.973 | 143.4 | 67.44 | 9.022 |
| Std. Error | 3.727 | 7.689 | 0.6981 | 0.9911 | 47.80 | 22.48 | 3.007 |

To examine the titration of genipin, 0.4 mL of various concentrations of genipin were added to 0.8 mL of undiluted platelet lysate material. After a brief mix, 0.5 mL were added to wells of a 12-well tissue culture plate, and the plate was placed into a 37° C. incubator. The final concentrations of genipin were 0, 1.25, 2.5, 5, and 10 mg/mL. After several hours, there was a color change in the wells containing genipin plus the platelet lysate material. After about 24 hours, all wells containing both genipin and platelet lysate material were dark blue as it is presumed a blue product forms when genipin reacts with amino acids.

All of the wells contained a gel-like substance except for wells containing 0 and 1.25 mg/mL genipin. The gel-like material was washed with several changes of HEPES-BSS to remove the DMSO and any unreacted genipin. Then, mesenchymal stormal cells (MSCs) were plated into each well at 2.5×10³ cells/cm². The growth medium contained either 5% of the platelet lysate material or 0% of the platelet lysate material as a source of growth factors. After six days of growth at 37° C. and 5% CO₂, the results were as shown in Table 2.

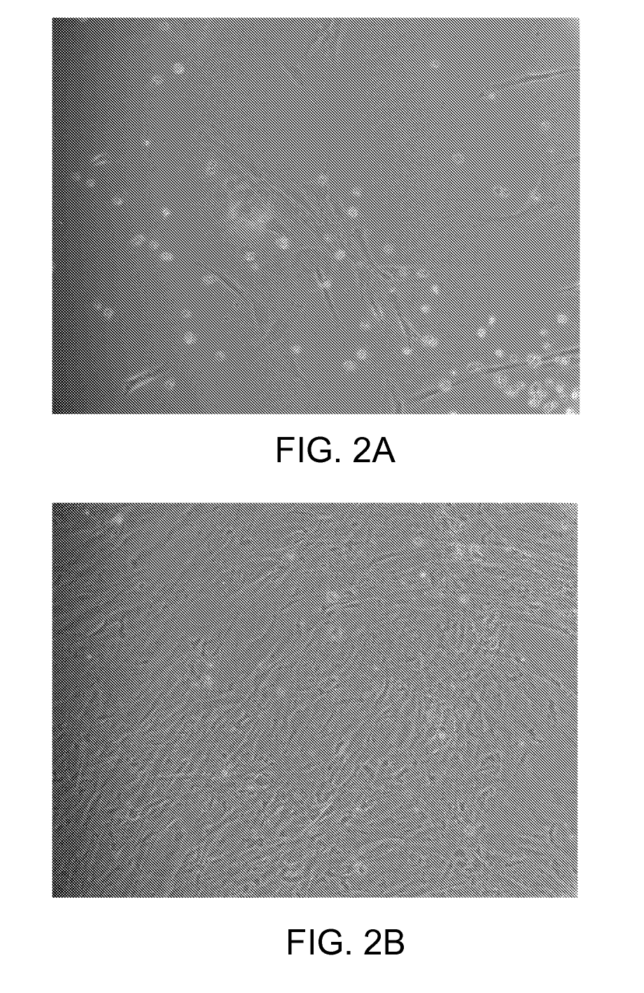

TABLE 2

| Well Contents | Medium | Growth |
|---|---|---|
| no gel* | 0% PL | None; cell attachment only (FIG. 2A) |
| no gel* | 5% PL | normal cell growth (FIG. 2B) |
| 2.5 mg/ml genipin | 0% PL | cell growth** |
| 2.5 mg/ml genipin | 5% PL | cell growth |

*no gel indicates that the well was only the standard tissue culture surface
**The cell proliferation in this well (0% PL in the medium) was less than that observed in the well containing medium with 5% PL

The dark blue color was too intense in the wells containing 5 and 10 mg/mL genipin to determine if any cells had attached and proliferated.

The cells appear to be growing on the surface of the gel, since the focal plane to view the cells was above the focal plane to view the cells in the wells with no gel (standard culture conditions).

These results demonstrate that cross-linked platelet lysate material retains the ability to supply growth factors for MSC growth.

In another experiment, the titration of platelet lysate material using a fixed concentration of 2.5 mg/mL genipin was assessed. Prior to adding the genipin, platelet lysate material was diluted with phosphate-buffered saline such that 60%, 80%, and 100% platelet lysate material was treated with genipin. A 12-well plate containing the various reaction mixtures was prepared as described above. After washing the formed gels, MSCs were plated as described above. Again, cells were cultured with either 0% or 5% platelet lysate material containing medium as the source of growth factors (Table 3).

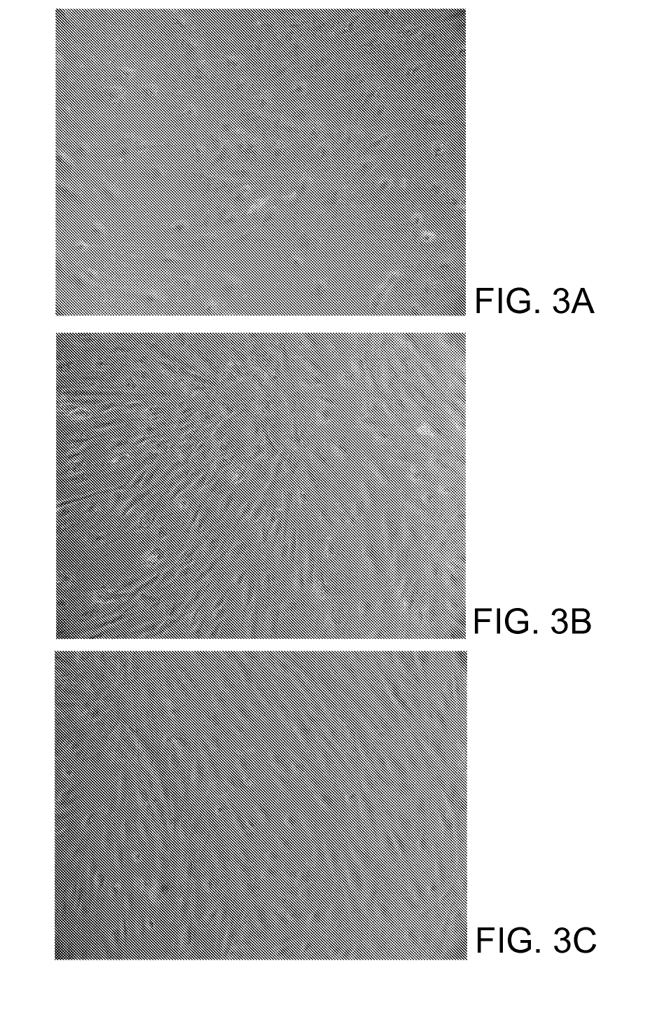

TABLE 3

| Well Contents | Medium | Growth |
|---|---|---|
| no gel* | 0% PL | none; cell attachment only |
| no gel* | 5% PL | normal cell growth |
| 60% PL | 0% PL | Growth (FIG. 3A) |
| 60% PL | 5% PL | Growth |
| 80% PL | 0% PL | Growth (FIG. 3B) |
| 80% PL | 5% PL | Growth |
| 100% PL | 0% PL | Growth (FIG. 3C) |
| 100% PL | 5% PL | Growth |

*no gel indicates that the well included only the standard tissue culture surface.

Cells attached and proliferated in all conditions except for the uncross-linked platelet lysate material plus 0% PL containing growth medium. Qualitatively, the cells proliferated to a greater number as the percent platelet lysate material that was cross-linked and served as the source of growth factors increased from 60% to 100%.

These results demonstrate that cross-linked platelet lysate material can supply the needed growth factors for cell growth.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising platelet material cross-linked to genipin.

2. The composition of claim 1, wherein said platelet material is a lysed platelet preparation.

3. The composition of claim 2, wherein said lysed platelet preparation was filtered through a 0.45 μm filter.

4. The composition of claim 2, wherein said lysed platelet preparation was filtered through a 0.2 μm filter.

5. The composition of claim 2, wherein said lysed platelet preparation was filtered through a 0.45 μm filter and a 0.2 μm filter.

6. The composition of claim 1, wherein said platelet material comprises supernatant from centrifugation of lysed platelets.

7. The composition of claim 6, wherein said platelet material is platelets lysed via a freeze/thaw cycle.

8. The composition of claim 7, wherein said lysed platelets are platelets lysed via at least two freeze/thaw cycles.

9. The composition of claim 6, wherein said centrifugation comprises a force between 2000×g and 4000×g for between 15 and 45 minutes.

10. The composition of claim 6, wherein said centrifugation comprises a force of about 3000×g for about 30 minutes.

11. The composition of claim 1, wherein said platelet material comprises greater than 200 pg of VEGF polypeptide per mL.

12. The composition of claim 1, wherein said platelet material contains from about 20 mg to about 80 mg of total protein per mL.

13. The composition of claim 12, wherein said composition is prepared by combining said platelet material with said genipin.

14. The composition of claim 12, wherein said composition is prepared by combining a solution of between 5 percent and 100 percent of said platelet material with said genipin.

15. The composition of claim 12, wherein said composition is prepared by combining a solution of between 60 percent and 95 percent of said platelet material with said genipin.

16. The composition of claim 12, wherein said composition is prepared by combining a solution of between 80 percent and 100 percent of said platelet material with said genipin.

17. The composition of claim 12, wherein said composition is prepared by combining said platelet material with from about 1.5 mg to about 20 mg of said genipin per mL.

18. The composition of claim 12, wherein said composition is prepared by combining said platelet material with from about 2.5 mg to about 10 mg of said genipin per mL.

19. The composition of claim 12, wherein said composition is prepared by combining said platelet material with from about 2.0 mg to about 5.0 mg of said genipin per mL.

20. The composition of claim 1, wherein said composition is configured in the shape of a film or sheet.

21. The composition of claim 20, wherein the thickness of said composition is between 50 μm and 10 mm.

22. A composition comprising platelet material attached to a matrix, wherein genipin cross-links said platelet material to a surface of said matrix.

23. The composition of claim 22, wherein said platelet material is a lysed platelet preparation.

24. The composition of claim 23, wherein said lysed platelet preparation was filtered through a 0.45 μm filter.

25. The composition of claim 23, wherein said lysed platelet preparation was filtered through a 0.2 μm filter.

26. The composition of claim 23, wherein said lysed platelet preparation was filtered through a 0.45 μm filter and a 0.2 μm filter.

27. The composition of claim 22, wherein said platelet material comprises supernatant from centrifugation of lysed platelets.

28. The composition of claim 27, wherein said platelet material is platelets lysed via a freeze/thaw cycle.

29. The composition of claim 28, wherein said lysed platelets are platelets lysed via at least two freeze/thaw cycles.

30. The composition of claim 27, wherein said centrifugation comprises a force between 2000×g and 4000×g for between 15 and 45 minutes.

31. The composition of claim 27, wherein said centrifugation comprises a force of about 3000×g for about 30 minutes.

32. The composition of claim 22, wherein said platelet material comprises greater than 200 pg of VEGF polypeptide per mL.

33. The composition of claim 22, wherein said platelet material contains from about 30 mg to about 80 mg of total protein per mL.

34. The composition of claim 33, wherein said composition is prepared by combining said platelet material with said matrix, wherein said matrix is coated with said genipin.

35. The composition of claim 33, wherein said composition is prepared by combining a solution of between 5 percent and 100 percent of said platelet material with said matrix, wherein said matrix is coated with said genipin.

36. The composition of claim 33, wherein said composition is prepared by combining a solution of between 60 percent and 95 percent of said platelet material with said matrix, wherein said matrix is coated with said genipin.

37. The composition of claim 33, wherein said composition is prepared by combining a solution of between 80 percent and 100 percent of said platelet material with said matrix, wherein said matrix is coated with said genipin.

38. The composition of claim 33, wherein said composition is prepared by combining said platelet material with from about 1.5 mg to about 20 mg of said genipin per mL to form a mixture, and contacting said mixture to said matrix.

39. The composition of claim 33, wherein said composition is prepared by combining said platelet material with from about 2.5 mg to about 10 mg of said genipin per mL to form a mixture, and contacting said mixture to said matrix.

40. The composition of claim 33, wherein said composition is prepared by combining said platelet material with from about 2.0 mg to about 5.0 mg of said genipin per mL to form a mixture, and contacting said mixture to said matrix.

41. The composition of claim 22, wherein said composition is configured in the shape of an esophageal segment.

42. The composition of claim 22, wherein said composition is a bandage for wound healing, an implantable esophageal segment, or a mucosal replacement device.

43. A method for making a composition comprising platelet material and genipin, wherein said method comprises contacting said platelet material with genipin, wherein said platelet material cross-links to said genipin.

44. A method for making a composition comprising a matrix, platelet material, and genipin, wherein said method comprises attaching said platelet material to said matrix via genipin.

45. A method for repairing an esophagus within a mammal, wherein said method comprising implanting an tubular tissue scaffold into said mammal in a position that bridges a gap in said esophagus, wherein said tubular tissue scaffold comprises platelet material.

46. The method of claim 45, wherein said tubular tissue scaffold comprises genipin.

47. The method of claim 45, wherein said platelet material is cross-linked to said tubular tissue scaffold via genipin.

48. The method of claim 45, wherein said tubular tissue scaffold comprises nanofibers.

49. A method for healing a wound, wherein said method comprising contacting said wound with a matrix comprising platelet material attached to said matrix via genipin.

50. The method of claim 49, wherein said matrix is a bandage.
